(19) United States
(12) Patent Application Publication
(10) Pub. No.: US 2026/0151127 A1
(43) Pub. Date: Jun. 4, 2026

(54) SUTURE PROCESSING MACHINE AND METHOD USING ULTRASONIC WAVES

(71) Applicant: HYUNDAE MEDITECH Co., Ltd., Wonju-si (KR)

(72) Inventor: Mi Hee SONG, Wonju-si (KR)

(73) Assignee: HYUNDAE MEDITECH Co., Ltd., Wonju-si (KR)

(21) Appl. No.: 19/460,444

(22) Filed: Jan. 27, 2026

Related U.S. Application Data
(62) Division of application No. 18/531,113, filed on Dec. 6, 2023.

(30) Foreign Application Priority Data
Sep. 11, 2023 (KR) 10-2023-0120579

(51) Int. Cl. A61B 17/06; D02G 3/44; D02J 3/02; A61B 17/00

(52) U.S. Cl. CPC A61B 17/06166; D02G 3/448; D02J 3/02; A61B 2017/00526; A61B 2017/06176

(57) ABSTRACT

The present disclosure provides a suture processing machine using ultrasonic waves, the suture processing machine including: a supply roll around which a suture to be processed is wound; a distal-end transferer configured to grip and transfer a distal end of the suture supplied from the supply roll; a tension controller configured to control tension of the suture between the distal-end transferer and the supply roll; a mold having an intagliated pattern to form cogs on the suture; both-end transferers installed at both sides of the mold to move the suture to the intagliated pattern of the mold or separate the suture from the intagliated pattern between the supply roll and the distal-end transferer; an ultrasonic wave generator configured to come in contact with the suture in the mold and transmit ultrasonic waves thereto; a first blade portion configured to remove a part of the suture that protrudes to an upper surface of the mold; a separation roller configured to support the suture from a lower side and assist in separation of the suture while the suture is being separated from the intagliated pattern by the both-end transferers; and a second blade portion configured to cut a tip of the suture transferred from the distal-end transferer.

### CROSS-REFERENCE TO RELATED APPLICATION

This application is a division of U.S. Application No. 18/531,113 filed on December 6, 2023, which claims priority to and the benefit of Korean Patent Application No. 2023-0120579, filed on September 11, 2023, the disclosure of which is incorporated herein by reference in its entirety.

### BACKGROUND

### Field of the Invention

The present disclosure relates to a suture processing machine and method using ultrasonic waves, and more particularly, to a suture processing machine and method for forming cogs on a suture used in plastic surgery or cosmetic treatment by using ultrasonic waves.

### Discussion of Related Art

In recent years, sutures for suturing a laceration of the skin or an organ have also been widely used for the purpose of lifting sagging skin or improving wrinkles.

In the past, a suture made of a material such as polydioxanone (PDO) that dissolves in the skin was inserted subcutaneously to induce collagen formation, but nowadays, a suture having cogs formed on a surface thereof is used to pull the skin or improve wrinkles by the cogs pulling subcutaneous tissue.

Cogs are also referred to as protrusions or barbs.

In order to form cogs on a surface of a suture, a method of forming cuts on the surface of the suture is mostly used, which has a problem in that the suture may break due to a reduction in a cross-section of the suture that is caused by the formation of cuts.

In order to address such problems, methods of forming cogs on a surface of a suture by inserting the suture in a mold and heating and pressing the suture have been proposed.

In this case, it is not easy to control a temperature in the mold, and when separating the suture from the mold after forming the cogs on the suture, it is difficult to maintain fine shapes of sites where the cogs are formed.

In order to address such problems, Korean Patent Registration No. 10-1921980 discloses forming cogs (11) on a surface of a suture (10) as in FIG. 2 by heating the suture using ultrasonic waves. When the suture is instantaneously heated using ultrasonic waves, the cogs can be formed on the suture even when the suture is not heated to a melting point of PDO, and thus durability of the suture is not reduced.

In the related art, as illustrated in FIG. 1, the suture (10) is transferred in a roll-to-roll manner, a mold (200) is lifted to place the suture (10) in the mold (200), and an ultrasonic wave generator (300) heats and presses the suture (10) in the mold (200).

While the suture is treated with ultrasonic waves, a central portion of the suture is elastically deformed due to tension acting between the rolls, and a surface portion of the suture is melted such that the cogs are formed and plastic flow occurs thereon. When the suture is separated from the mold and cooled, residual stress is generated between the central portion and the surface portion of the suture, and the residual stress causes the quality of the suture to be non-uniform.

Also, a problem of cooling the mold according to repeated treatment and an overflow of the suture that occurs when the suture is pressed also cause the quality of the suture to be defective.

### Related Art Document

### Patent Document

### KR 10-1921980 B1, November 27, 2018

### SUMMARY OF THE INVENTION

The present disclosure is for addressing the above-described problems of the related art and is particularly directed to preventing the presence of residual stress in a suture and improving the quality of the suture.

The present disclosure provides a suture processing machine using ultrasonic waves, the suture processing machine including: a supply roll around which a suture to be processed is wound; a distal-end transferer configured to grip and transfer a distal end of the suture supplied from the supply roll; a tension controller configured to control tension of the suture between the distal-end transferer and the supply roll; a mold having an intagliated pattern to form cogs on the suture; both-end transferers installed at both sides of the mold to move the suture to the intagliated pattern or separate the suture from the intagliated pattern between the supply roll and the distal-end transferer; an ultrasonic wave generator configured to come in contact with the suture in the mold and transmit ultrasonic waves thereto; a first blade portion configured to remove a part of the suture that protrudes to an upper surface of the mold; a separation roller configured to support the suture from a lower side and assist in separation of the suture while the suture is being separated from the intagliated pattern by the both-end transferers; and a second blade portion configured to cut a tip of the suture transferred from the distal-end transferer.

The supply roll and the tension controller may be integrally formed.

The suture processing machine may further include a cooler configured to cool the mold.

The second blade portion may further include a grip portion configured to grip the suture transferred from the distal-end transferer.

The suture processing machine may further include a tray portion configured to collect the suture cut by the second blade portion.

The present disclosure provides a suture processing method using ultrasonic waves, the suture processing method including: step 1 of receiving a suture from a supply roll and gripping the suture by a distal-end transferer; step 2 of transferring the suture into an intagliated pattern of a mold positioned between the supply roll and the distal-end transferer; step 3 of removing tension of the suture between the supply roll and the distal-end transferer; step 4 of forming cogs on the suture by coupling an ultrasonic wave generator to the mold and transmitting ultrasonic waves to the suture; step 5 of separating the ultrasonic wave generator and removing a part of the suture that protrudes to an upper surface of the mold by using a first blade portion; step 6 of adding tension to the suture between the supply roll and the distal-end transferer; step 7 of separating the suture from the mold; and step 8 of transferring the suture having the cogs formed thereon to a second blade portion to cut a tip of the suture.

In step 2, the suture may be moved toward the mold by both-end transferers disposed at both sides of the mold to insert the suture into the intagliated pattern of the mold, and here, the tension may be controlled so as not to increase.

In step 7, the suture may be moved upward by the both-end transferers disposed at both sides of the mold.

In step 7, the suture may be moved upward by the both-end transferers disposed at both sides of the mold, and a separation roller may move between the suture and the upper surface of the mold.

In step 8, a distal end of the suture having the cogs formed thereon that is transferred by the distal-end transferer may be transferred to the second blade portion and gripped, the distal-end transferer moving toward the tip of the suture again may grip the tip of the suture and transfer the tip of the suture to the second blade portion, and then the second blade portion may cut the tip.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a view illustrating a suture processing machine using ultrasonic waves of the related art;

FIG. 2 is a view illustrating the shape of a suture having cogs formed thereon according to the related art;

FIG. 3 is a view illustrating a suture processing machine using ultrasonic waves of the present disclosure; and

FIGS. 4 to 10 are views illustrating an order of operation of the suture processing machine of the present disclosure.

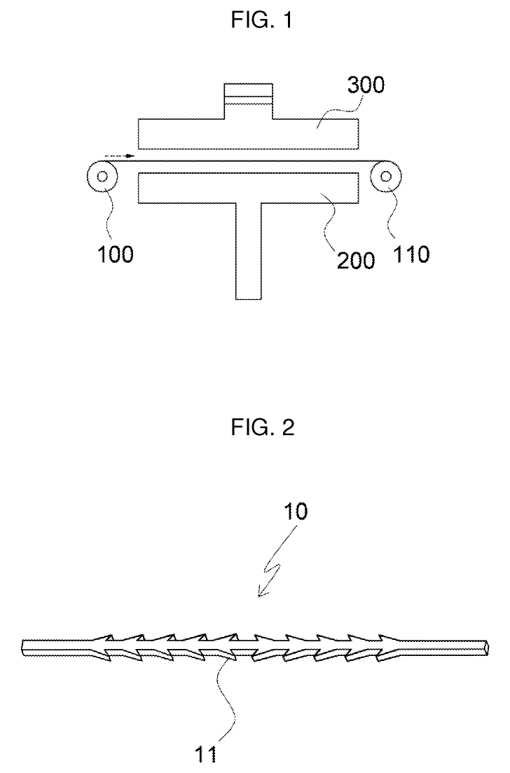

### DETAILED DESCRIPTION OF EXEMPLARY EMBODIMENTS

Hereinafter, the present disclosure will be described in more detail according to embodiments thereof with reference to the accompanying drawings.

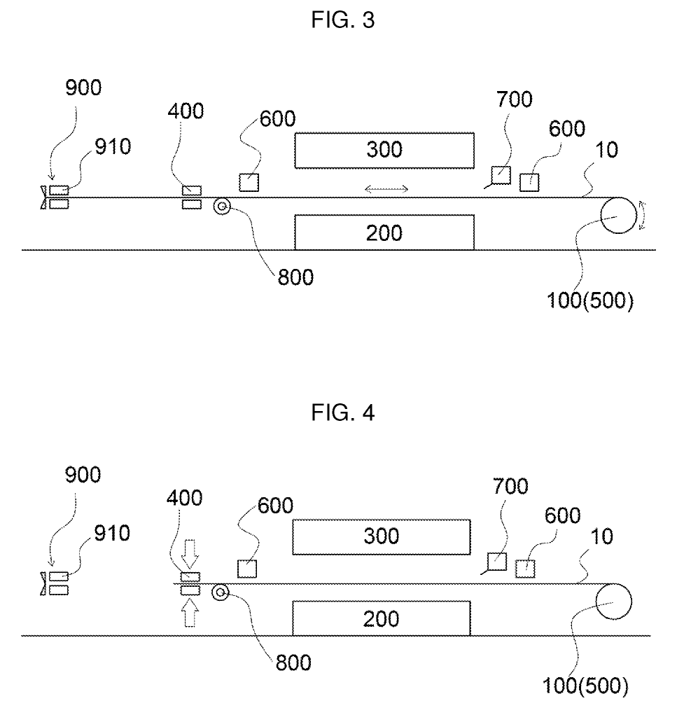

FIG. 3 is a view illustrating a suture processing machine using ultrasonic waves of the present disclosure.

The suture processing machine using ultrasonic waves of the present disclosure includes: a supply roll 100 around which a suture 10 to be processed is wound; a distal-end transferer 400 configured to grip a distal end of the suture 10 supplied from the supply roll 100 and transfer the suture 10; a tension controller 500 configured to control tension of the suture 10 between the distal-end transferer 400 and the supply roll 100; a mold 200 having an intagliated pattern to form cogs on the suture 10; both-end transferers 600 installed at both sides of the mold 200 to move the suture to the intagliated pattern of the mold 200 or separate the suture from the intagliated pattern between the supply roll 100 and the distal-end transferer 400; an ultrasonic wave generator 300 configured to come in contact with the suture in the mold 200 and transmit ultrasonic waves thereto; a first blade portion 700 configured to remove a part of the suture that protrudes to an upper surface of the mold 200; a separation roller 800 configured to support the suture from a lower side and assist in separation of the suture while the suture is being separated from the intagliated pattern by the both-end transferers 600; and a second blade portion 900 configured to cut a tip of the suture 10 transferred from the distal-end transferer 400.

The suture 10 supplied from the supply roll 100 may be made of any material as long as the material allows plastic flow to occur on the suture 10 and allows cogs to be formed on the suture 10 when the suture 10 is heated and pressed in the mold.

A distal end of the suture 10 unwound from the supply roll 100 is gripped by the distal-end transferer 400 (step 1) as in FIG. 4, and a certain magnitude of tension is maintained on the suture 10 by the tension controller 500.

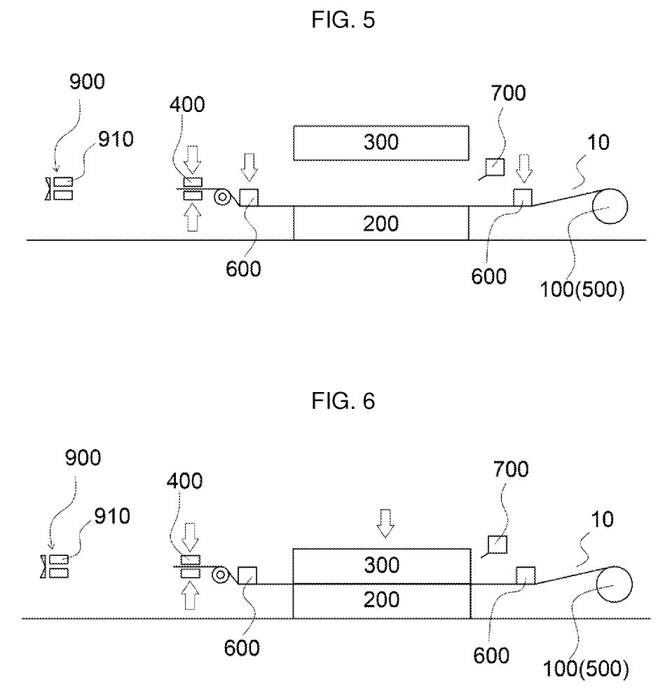

Then, in order to insert the suture 10 gripped by the distal-end transferer 400 into the mold, as in FIG. 5, the both-end transferers 600 move the suture 10 toward the mold (downward) to insert the suture into an intagliated pattern (not illustrated) of the mold 200 (step 2).

The both-end transferers 600 are disposed at both sides of the mold to move the suture toward the mold (downward) or separate the suture from the mold.

The tension controller 500 controls tension so that excessive tension does not act when the suture is inserted into the mold by the operation of the both-end transferers 600. The tension controller 500 and the supply roll 100 are integrally formed so that tension can be controlled by further supplying the suture of a certain length from the supply roll 100 or rewinding the suture of a certain length around the supply roll 100. Of course, a separate sensor that can detect tension is provided in the tension controller.

When the transfer of the suture into the intagliated pattern is completed, the tension controller 500 removes the tension acting on the suture (step 3).

Then, as in FIG. 6, the ultrasonic wave generator 300 moves downward and comes in contact with the suture 10 in the intagliated pattern of the mold 200 to transmit ultrasonic waves to the suture 10. The temperature of the suture increases due to the suture receiving ultrasonic energy, and parts of the suture that melt as the temperature rises cause flow due to pressure of the ultrasonic wave generator pressing the suture and fill the intagliated pattern of the mold such that cogs are formed (step 4). Here, a frequency of the ultrasonic waves to be applied is about 2 kHz to 4 kHz, but the range of the frequency may be controlled according to the thickness or material of the suture.

When the formation of the cogs is completed, the ultrasonic wave generator 300 is separated from the mold 200.

Here, a cooler configured to spray air to the ultrasonic wave generator and the mold may be provided to cool the ultrasonic wave generator and the mold.

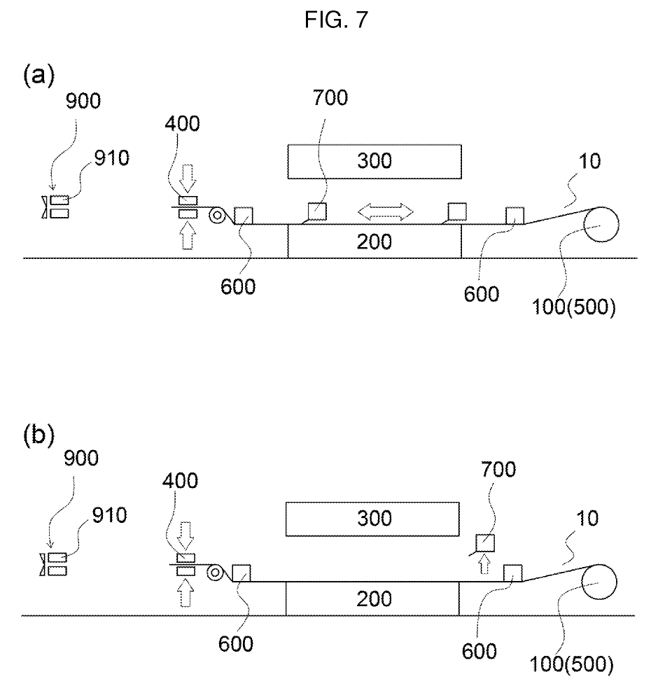

When the ultrasonic wave generator 300 is coupled to the mold 200 and presses the suture 10, the melted suture leaks between the ultrasonic wave generator 300 and the mold 200 in some cases, and the leaked part of the suture protrudes to the upper surface of the mold 200 after the ultrasonic wave generator 300 is separated. The first blade portion 700 moves downward and moves while in contact with the upper surface of the mold 200 to remove the part of the suture that protrudes to the upper surface of the mold 200, and then the first blade portion 700 returns to its original position (step 5, FIG. 7).

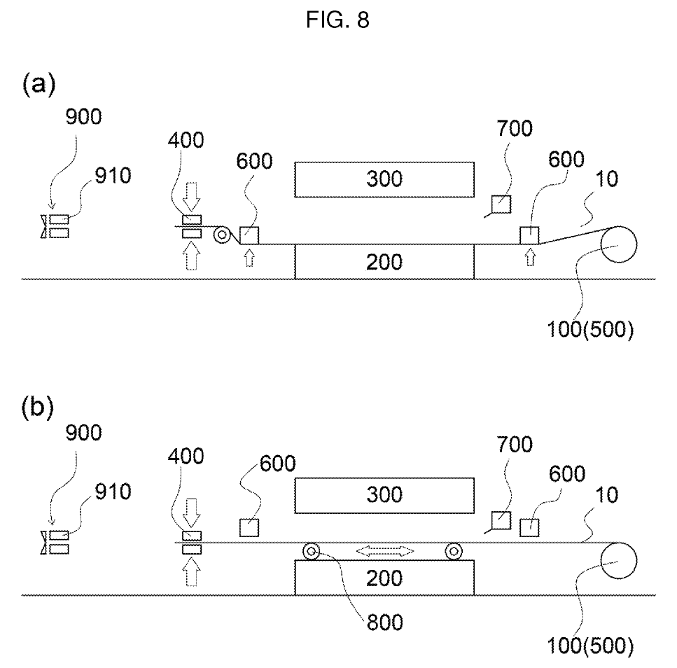

Then, the tension controller 500 causes tension to act on the suture 10 (step 6), and the both-end transferers 600 move upward and lift both ends of the suture to separate the suture 10 from the mold 200 (step 7, FIG. 8A). Here, when the separation roller 800 moves between the suture 10 and the upper surface of the mold 200, the suture 10 may be more reliably separated from the mold 200 by the separation roller 800 (FIG. 8B).

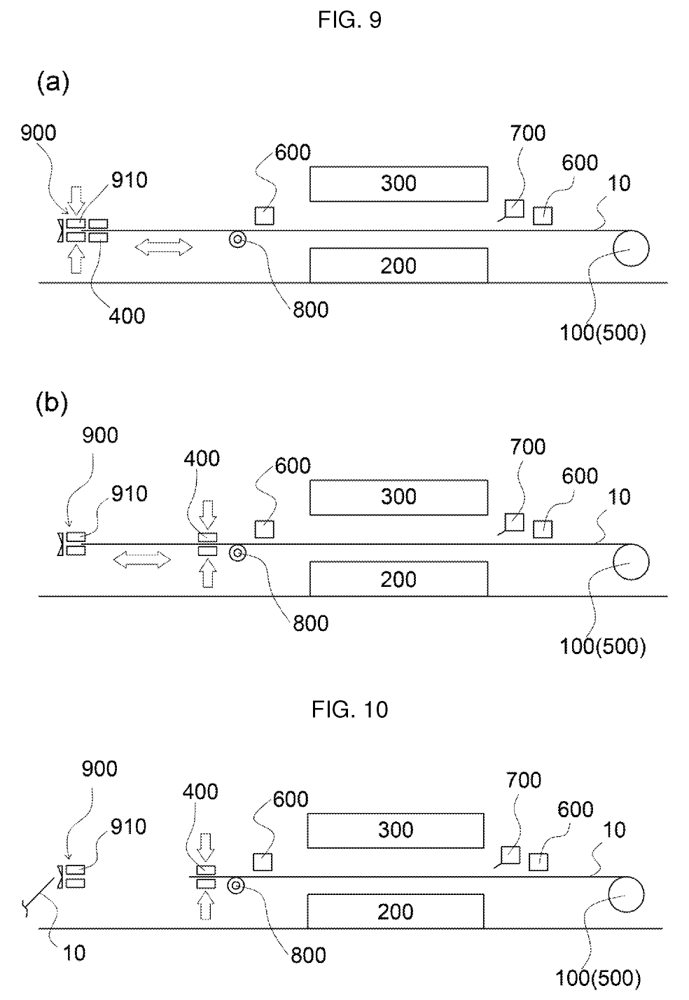

The distal end of the suture separated from the mold 200 is transferred to the second blade portion 900 by the distal-end transferer 400 gripping the distal end (FIG. 9A). Here, the suture is unwound from the supply roll. After the distal end of the suture 10 is gripped by a grip portion 910 of the second blade portion 900, the distal-end transferer 400 moves to a tip of the suture, grips the tip (FIG. 9B), and moves the tip to the second blade portion 900. Here, of course, the grip portion 910 does not grip the distal end of the suture.

Then, the second blade portion 900 cuts the tip of the suture, and the suture whose tip is cut is collected by a tray portion (not illustrated).

As the distal-end transferer 400 returns to its original position, the above-described process restarts from step 1, and in the entire process in which the suture is moved by the distal-end transferer 400, the suture is unwound from or rewound around the supply roll, and tension on the suture is controlled by the tension controller 500.

The present disclosure has advantageous effects that it is possible to prevent the presence of residual stress in a suture and improve the quality of the suture.

The present disclosure has been described in more detail above based on the drawings so that those of ordinary skill in the art can easily practice the present disclosure. All terms including technical or scientific terms used herein have the same meaning as commonly understood by those of ordinary skill in the art to which the present disclosure pertains. Terms, such as those defined in commonly used dictionaries, should be interpreted to have meanings consistent with their meanings in the context of the relevant art and should not to be construed in an idealized or overly formal sense unless expressly so defined herein.

What is claimed is:

1. A suture processing method using ultrasonic waves, the suture processing method comprising:
step 1 of receiving a suture from a supply roll and gripping the suture by a distal-end transferer;
step 2 of transferring the suture into an intagliated pattern of a mold positioned between the supply roll and the distal-end transferer;
step 3 of removing tension of the suture between the supply roll and the distal-end transferer;
step 4 of forming cogs on the suture by coupling an ultrasonic wave generator to the mold and transmitting ultrasonic waves to the suture;
step 5 of separating the ultrasonic wave generator and removing a part of the suture that protrudes to an upper surface of the mold by using a first blade portion;
step 6 of adding tension to the suture between the supply roll and the distal-end transferer;
step 7 of separating the suture from the mold; and
step 8 of transferring the suture having the cogs formed thereon to a second blade portion to cut a tip of the suture.

2. The suture processing method of claim 1, wherein, in step 2, the suture is moved toward the mold by both-end transferers disposed at both sides of the mold to insert the suture into the intagliated pattern of the mold, and here, the tension is controlled so as not to increase.

3. The suture processing method of claim 1, wherein, in step 7, the suture is moved upward by the both-end transferers disposed at both sides of the mold.

4. The suture processing method of claim 1, wherein, in step 7, the suture is moved upward by the both-end transferers disposed at both sides of the mold, and a separation roller moves between the suture and the upper surface of the mold.

5. The suture processing method of claim 1, wherein, in step 8, a distal end of the suture having the cogs formed thereon that is transferred by the distal-end transferer is transferred to the second blade portion and gripped, the distal-end transferer moving toward the tip of the suture again grips the tip of the suture and transfers the tip of the suture to the second blade portion, and then the second blade portion cuts the tip.
